(19) United States
(12) Patent Application Publication
(10) Pub. No.: US 2017/0224753 A1
(43) Pub. Date: Aug. 10, 2017

(54) Homogeneous Composition of Plant-Based Oils and Hydrosols

(71) Applicants: Joshua C Woodcook, Cedar Hill, UT (US); Ronald L Spence, Eagle Mountain, UT (US)

(72) Inventors: Joshua C Woodcook, Cedar Hill, UT (US); Ronald L Spence, Eagle Mountain, UT (US)

(21) Appl. No.: 15/016,052

(22) Filed: Feb. 4, 2016

(51) Int. Cl. A61K 36/61

(52) U.S. Cl. CPC A61K 36/61; A61K 2236/31; A61K 2236/37

(57) ABSTRACT

A homogeneous composition of an essential oil and essence water is provided. The essential oil may be derived from the plant matter through an extraction process, such as steam-based distillation. The essence water may be a byproduct of the extraction of the essential oil from plant matter. The homogeneous composition may be formed by breaking down the components of the essential oil into small particles and distributing them evenly throughout the essence water to form a colloid. The essential oil and the essence water may be derived from the same plant matter or different plant matter. The homogeneous composition may be topically applied or ingested for therapeutic or nutritional benefits.

### CROSS-REFERENCE TO RELATED APPLICATIONS

None.

### STATEMENT REGARDING FEDERALLY SPONSORED RESEARCH OR DEVELOPMENT

Not Applicable.

### BACKGROUND OF THE INVENTION

1. The Field of the Invention

The present invention relates generally to the complex processing of organic plant matter, and more particularly, but not necessarily entirely, the processing of essential oils and essence water derived from plant matter.

2. Description of Related Art

An essential oil is a concentrated hydrophobic liquid that may contain volatile aroma compounds from plants. Essential oils are also known as volatile oils, ethereal oils, aetherolea, or simply as the oil of the plant from which they were extracted, such as oil of clove. The oil is considered “essential” in the sense that it may contain the “essence of” the plant's fragrance—the characteristic fragrance of the plant from which it is derived.

Essential oils have been used therapeutically for centuries and recent research studies on essential oils show positive effects for a variety of health concerns. Essential oils can be inhaled, applied topically, incorporated into massage, or taken internally. Increasingly, essential oils are used to increase emotional states and physical wellness. Essential oils are also known to be effective as stress relievers. In addition, essential oils and extracts have been found to contain antiseptic, antimicrobial, anti-inflammatory and antiviral agents. Some research has revealed that plant-based phenols, flavonoids, isoflavones, terpenes, glucosinolates and other compounds present in fruits, vegetables and leafy herbs possess antioxidant and anti-carcinogenic properties.

Essential oils may be extracted from plants using a wide variety of processes. One of the most common methods of extracting essential oils is distillation. The distillation process may include placing plant matter into a distillation apparatus over water. As the water is heated, the steam passes through the plant matter, vaporizing the volatile compounds. The steam carrying the essential oils then flows through a coil, where they condense back to liquid, which is then collected in a collection chamber. The essential oils and water are then separated by collecting the oils from the top surface of the distillate in the collection chamber.

The separated and recondensed water is sometimes referred to as a hydrosol, hydrolat, distillate or plant water essence or, as referred to herein, essence water. The essence water may contain the same volatile compounds found in the essential oils, but in much smaller and milder concentrations, and may be a colloidal suspension of these compounds along with water-soluble components obtained from the steam distillation of the plant matter. Further, the components in the essence water may be primarily acids, which are hydrophilic (water-loving). In most cases, the essence water is simply discarded as a by-product of the essential oil extraction process—although it is sometimes marketed as a separate product, such as a fragrance, flavoring, or therapeutic treatment. It would, however, be an improvement over the prior art to provide a new and useful purpose for essence water in combination with essential oils.

The features and advantages of the invention will be set forth in the description which follows, and in part will be apparent from the description, or may be learned by the practice of the invention without undue experimentation. The features and advantages of the invention may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a diagram of a system suitable for extracting an essential oil from plant matter and creating essence water according to an embodiment of the present disclosure;

FIG. 2. is a diagram of a homogenizer for creating a homogeneous mixture of an essential oil and essence water according to an embodiment of the present disclosure;

FIG. 3 is a diagram of a homogenizer for creating a homogeneous mixture of an essential oil and essence water according to an embodiment of the present disclosure; and

FIG. 4 is a flow diagram of a process for creating a homogeneous mixture of an essential oil and essence water according to an embodiment of the present disclosure.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

In describing and claiming the present disclosure, the following terminology will be used in accordance with the definitions set out below.

As used in this specification and the appended claims, the singular forms “a,” “an,” and “the” include plural referents unless the context clearly dictates otherwise. As used herein, the terms “comprising,” “including,” “containing,” “characterized by,” “having,” and grammatical equivalents thereof are inclusive or open-ended terms that do not exclude additional, unrecited elements or method steps.

As used herein, the term “essential oil” may refer to a plant-based oil, including any oil extracted or derived from plant matter through distillation or some other process. As used herein, the term “essence water” may refer to an aqueous-based solution containing compounds derived from plant matter through distillation or some other process. In an embodiment, the essence water is formed as a byproduct of a process utilized to separate essential oil from plant matter. For example, the essence water may be formed from re-condensed steam used in a distillation process to extract essential oils.

In an embodiment, the present invention may include a homogeneous mixture of an essential oil and essence water. In an embodiment, the essential oil and essence water may be derived from the same type of plant. In an embodiment, the essential oil and essence water may be derived from different types of plant.

In an exemplary embodiment, the plant matter used for the oils and essence water is from a plant genus selected from Origanum, Mentha, Agastache, Ocimum, Salvia, Artemisia, Melissa, Achillea, Thymus, Coridothymus, Micromeria, Rosmarinus, Pelargonium and Coleus. In an exemplary embodiment, the plant matter is from Mentha spicata, Mentha piperita, Rosmarinus officinalis, Origanum syriacum, Origanum dayi, Origanum vulgare, Thymus vulgare, Ocimum basilicum or Melissa officinalis. In an exemplary embodiment, suitable plants include, but are not limited to, angelica root, anise, balsam, basil, bay, bay laurel, beeswax, benzoin, bergamot, bergamot mint, bois-de-rose, boronia, cajeput, cannabis, cardamum, carrot seed, cedarwood, chamomile, cinnamon, citronella, clary sage, clove bud, coriander, cypress, dill, elemi, eucalyptus (including lemon eucalyptus), fennel, fir needle, galbanum, geranium (including rose geranium), ginger, grapefruit, helichrysum, hyssop, immortelle, jasmine, juniper berry, kanuka, lavender, lavendin, lemon, lemongrass, lime, linden blossom, mandarin, manuka, marjoram, may chang, myrrh, myrtle (including lemon myrtle), neroli, niaouli, nutmeg, oakmoss, olibanum orange (bitter and sweet), oregano, palmarosa, parsley, patchouli , pepper (black, white, sweet or hot), peppermint and related mints, petitgrain pine, ravensara, rose, rosemary, rosewood, sandalwood, spearmint, spikenard, spruce, tangerine, tea tree (common, lemon, New Zealand), thyme, tobacco, tuberose, vanilla, vetiver, violet leaf, yarrow and ylang ylang.

In an embodiment, plants from the Labiaceae, Umbelliferae and Compositae (Apiaceae) families are suitable for use in the present invention. In an embodiment, the plants are from the mint family or other aromatic herbs, e.g., oregano and rosemary, to name a few. In an embodiment, suitable plants include, but are not limited to, Origanum spp. (e.g., O. syriacum, O. dayi, O. vulgare, O. onites), Mentha spp. (e.g., M. longifolia, M. spicata, M. piperita) Agastache, Ocimum spp. (e.g., O. basilicum, O. gratissimum, O. canum, O. americanum), Salvia spp. (e.g., S.qfficinalis, S fruticos\acute{α}), Artemisia spp. (e.g., A. dranunculus, A. judaica, A. aborescens, A. absinthum), Melissa officinalis, Achillea spp. (e.g., A. millenfolium, A. fragrantissimum), Thymus vulgare, Coridothymus capitatus, Micromeria fruticosa, Rosmarinus officinalis, Pelargonium graveolens (geranium) and Coleus spp. In exemplary embodiments, mint (e.g., Mentha spicata, M. piperita), rosemary (Rosmarinus officinalis), oregano (Origanum syriacum, O. dayi, O. vulgare), thyme (Thymus vulgare), Ocimum basilicum and Melissa officinalis are used.

In an embodiment, any part of the plant is suitable for use in the present invention. This includes, but is not limited to, flowers, seeds, bracts and other floral parts, stems, leaves and roots. In an embodiment, leaves and stems are utilized. In an embodiment, the woody parts of a plant are utilized.

In an embodiment, the present invention may include combining an essential oil and essence water using a homogenization process. In an embodiment, the present invention may include processing the essential oil into a state consisting of extremely small particles distributed uniformly throughout the essence water. In an embodiment, a composition according to the present invention may be accomplished by reducing the size of the essential oil globules and disbursing them throughout the essence water using a centrifuge or some other device. In an embodiment, a composition according to the present invention may be accomplished by reducing the size of the essential oil globules and disbursing them throughout the essence water by placing the essential oil under high pressure.

In an embodiment, the present invention may include a homogenized composition of essential oil and essence water that suitable for topical application on a human for therapeutic purposes. In an embodiment, the composition may have an increased absorption rate as compared to an essential oil alone. In an embodiment, the present invention may include a homogenized composition of an essential oil and essence water that is suitable for human ingestion. In an embodiment, the present invention may include a homogenized composition of an essential oil and essence water that is suitable for use as a food additive.

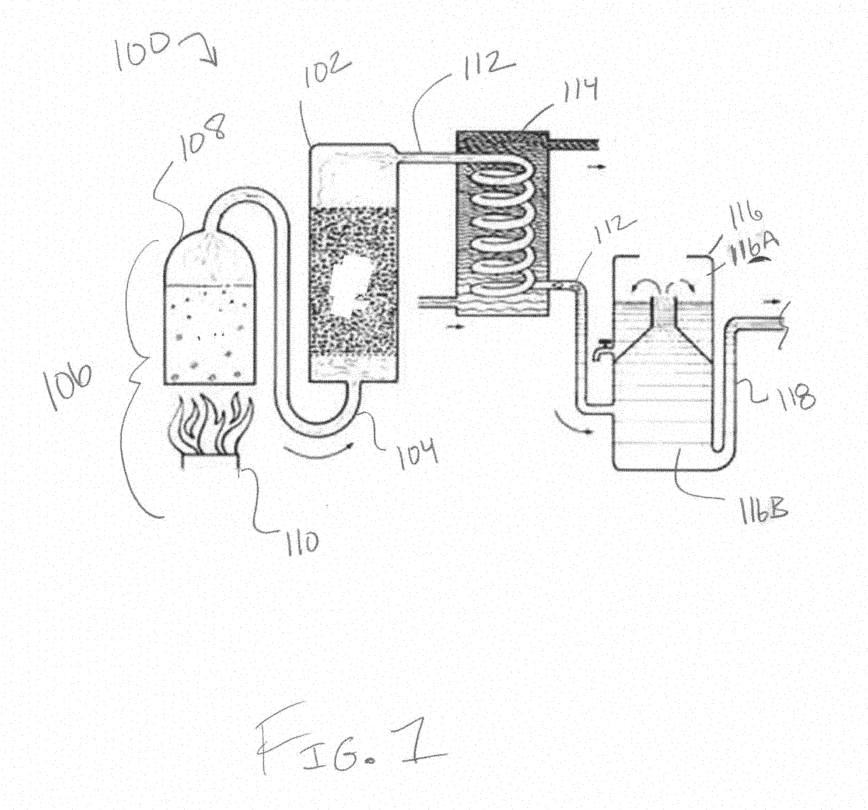

Referring now to FIG. 1, there is depicted generally a system 100 for extracting essential oils and essence water from plant matter according to an embodiment of the present disclosure. It will be appreciated that the plant matter can be derived from any type of plant, including, but not limited to, those identified above. In an embodiment, the system 100 includes a distillation chamber 102 having the desired plant matter from which the essential oil will be extracted disposed therein. The distillation chamber 102 may be connected to an input pipe 104. The input pipe 104 may direct steam from a steam generator 106 into the distillation chamber 102. The steam generator 106 may include a container 108 having water disposed therein.

A heat source 110 may be utilized to heat the water within the container 108 in order to generate the necessary steam. The distillation chamber 102 may be connected to an output pipe 112. The output pipe 112 may include a coiled portion that passes through a cooling chamber 114. The cooling chamber 114 may be fed with a supply of cooled water. After the cooling chamber 114, the output pipe 112 is fed into a separation device 116 that is operable to separate the essential oils from the essence water.

In operation, the system 100 directs steam into the distillation chamber 102 where it passes through the plant matter. The steam, along with the essential oils, are passed through the output pipe 112 into the cooling chamber 114. The steam re-condenses inside the cooling chamber 114 and is fed into the separation device 116. The essential oils, being lighter than water, separate from the essence water and are collected in a top portion 116A of the separation device 116. The essence water is directed from a bottom portion 116B of the separation device 116 through an output pipe 118 where it may be collected. Thus, it will be appreciated that operation of the system 100 produces both a supply of essential oils and essence water from the plant matter. It will be further appreciated that the system 100 may be utilized to provide a supply of essential oils and essence water from a plurality of different types of plants. It will be even further appreciated that the system 100 is an exemplary system according to the present invention, and that any system now known or known in the future that operates to separate essential oils and essence water from plant matter may be utilized in accordance with the principles of the present invention.

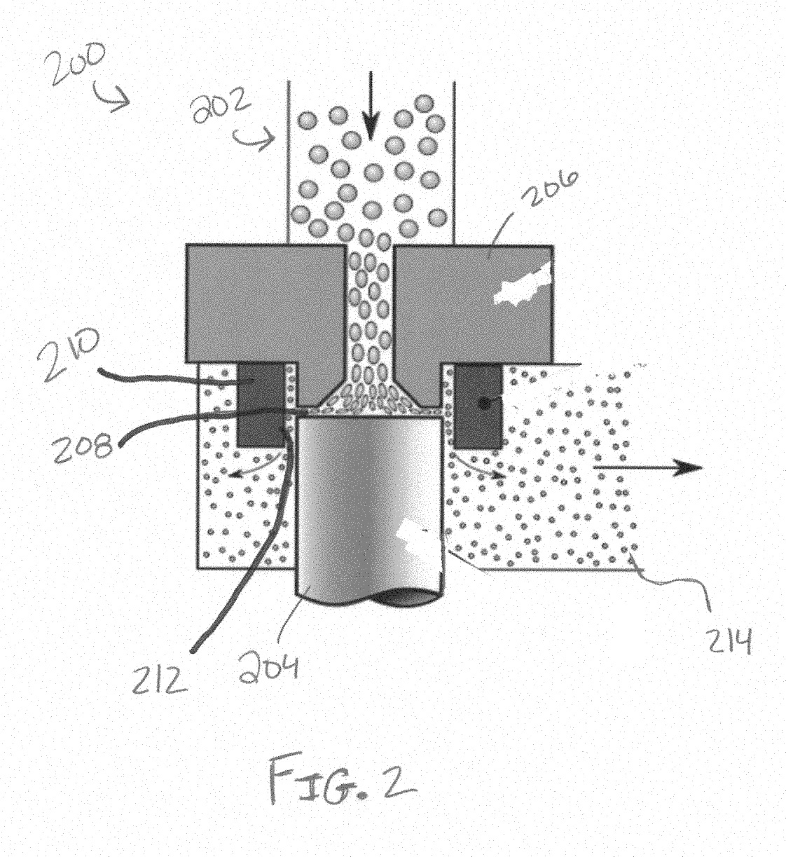

Referring now to FIG. 2, there is depicted a diagram of an exemplary system 200 for forming a homogeneous composition of essential oils and essence water according to an embodiment of the present disclosure. In particular, it will be appreciated that because the essential oils are a concentrated hydrophobic liquid that they will not easily mix with essence water. In an embodiment, the essential oils and essence water are derived from plant matter using the system 100 as shown in FIG. 1. It will be appreciated, however, that the essential oils and essence water may be derived using a different system than the one shown in FIG. 1. Further, the essential oils and the essence water may be derived from the same plant matter or different plant matter.

The system 200 comprises an input feed 202 that provides a fluid path for the essential oil and the essence water in an unmixed state, i.e., a non-homogenous mixture. The system 200 may further comprise a valve 204 and a seat 206 that define small jets 208. Disposed adjacent an outlet of the jets 208 is an impact ring 210 having an impact surface 212. The system 200 may further comprise an outlet chamber 214 fluidly interconnected to the input feed 202 via the jets 208. In operation, the essential oil and the essence water are forced from the input feed 202 into the jets 208 under high pressure. The essential oil and the essence water pass through the jets 208 and impact against the impact surface 212 of the impact surface 210, which impact causes the oil globules of the essential oil to be broken into smaller particles. The smaller and reduced in size particles of the essential oil then intermix with the essence water in the outlet chamber 214 to form a homogenous mixture. The homogenous mixture may then be placed in product containers.

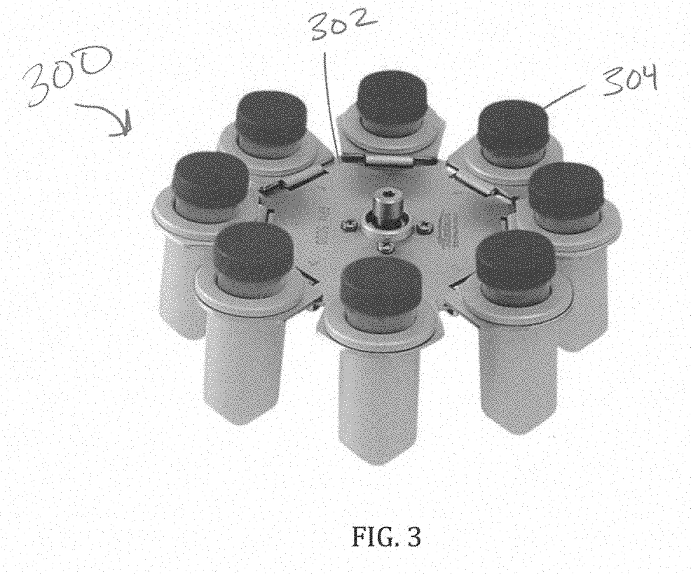

Referring now to FIG. 3, there is depicted a diagram of an exemplary system 300 for forming a homogeneous composition of an essential oil and essence water according to an embodiment of the present disclosure. The system 300 may comprise a centrifugal homogenizer 302 having a plurality of mixing tubes 304. In an embodiment, an essential oil and essence water are placed in the mixing tubes 304. The tubes 304 are then spun at high revolutions. The centrifugal homogenizer 302 uses centrifugal forces in generating the shearing forces required for breaking the globules of the essential oil into smaller particles and mixing the essential oil and essence water into a homogenous mixture.

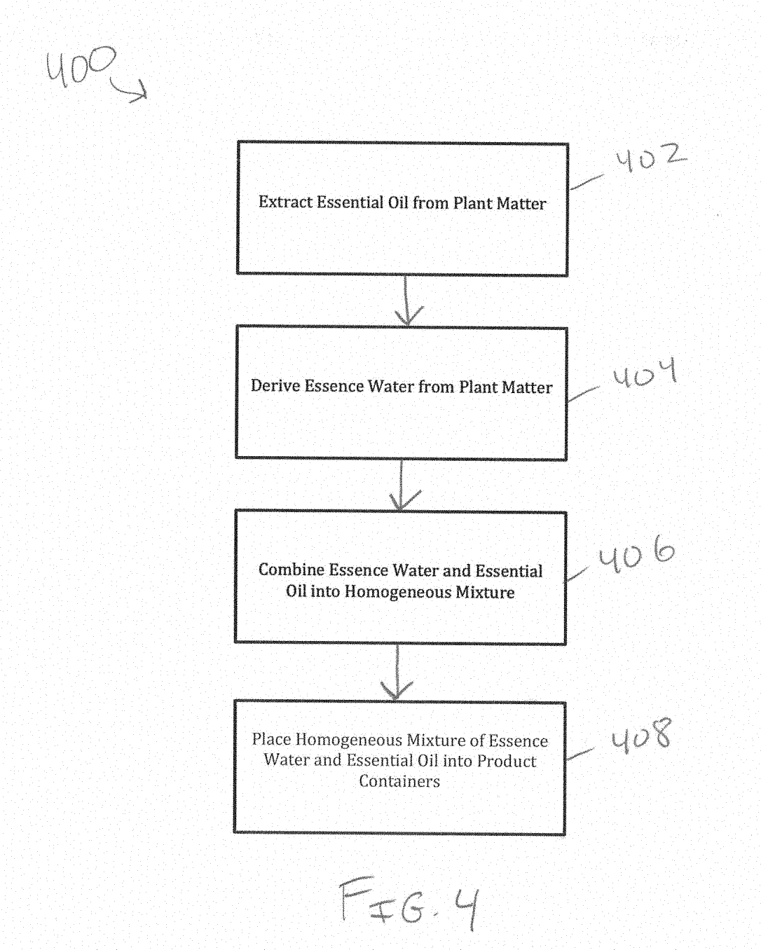

Referring now to FIG. 4, there is depicted a flow diagram of a process 400 for generating a homogeneous mixture of an essential oil and essence water according to an embodiment of the present disclosure. At step 402, the essential oil is extracted from plant matter. It will be appreciated that the plant matter may be any suitable plant matter that contains an essential oil. In an embodiment, the essential oil is extracted using a distiller and a steam-based distillation process. However, any process for extracting the essential oil from the plant matter may be utilized. At step 404, essence water is derived from plant matter. In an embodiment, the essence water is a by-product of a steam distillation process utilized to extract essential oil from plant matter. For example, the essence water may be separated from an essential oil from recondensed steam used in a distillation process. It will be appreciated that the essential oil and the essence water may be derived from the same type of plant matter or different types of plant matter. At step 406, the essential oil and the essence water are combined to form a homogeneous mixture by reducing the size of the particles of the essential oil into small particles. In an embodiment, the essential oil and the essence water are combined using a high-pressure homogenization process. In an embodiment, the essential oil and the essence water are combined using a centrifugal homogenization process. At step 408, the homogeneous mixture is placed into a product container.

It will be appreciated that a homogeneous mixture according to the present invention may include more than one essential oil. It will further be appreciated that a homogeneous mixture according to the present invention may include multiple essential oils from multiple different types of plants. It will still be further appreciated that a homogeneous mixture according to the present invention may include more than one type of essence water. It will further be appreciated that a homogeneous mixture according to the present invention may include essence water derived from multiple different types of plants.

In an embodiment, a homogeneous mixture according to the present disclosure may include essential oil and essence water according to the following percentages shown in Table 1, below.

TABLE 1

| Essential Oil | Essence Water |
|---|---|
| 10%-15% | 85%-90% |
| 15%-25% | 75%-85% |
| 25%-35% | 65%-75% |
| 35%-45% | 55%-65% |
| 45%-55% | 45%-55% |
| 55%-65% | 35%-45% |
| 65%-75% | 25%-35% |
| 75%-85% | 15%-25% |
| 85%-90% | 10%-15% |

According to the present disclosure, a composition for therapeutic treatment of a human being comprises: an essential oil derived from plant matter; and essence water derived from plant matter; wherein the essential oil and essence water form a homogeneous mixture. According to the present disclosure, the composition described above, wherein the essential oil and the essence water are derived from different types of plants. According to the present disclosure, the composition described above, wherein the essential oil and the essence water are derived from the same types of plant. According to the present disclosure, the composition described above, wherein the plant matter from which the essential oil is derived is an herb. According to the present disclosure, the composition described above, wherein the plant matter from which the essential oil is derived is a fruit. According to the present disclosure, the composition described above, wherein the plant matter from which the essential oil is derived is bark of a plant. According to the present disclosure, the composition described above, wherein the plant matter from which the essential oil is extracted is a grass. According to the present disclosure, the composition described above, wherein the plant matter from which the essential oil is extracted is eucalyptus. According to the present disclosure, the composition described above, wherein the plant matter from which the essential oil is extracted is one of a shrub and tree. According to the present disclosure, the composition described above, wherein the essence water is derived from an herb. According to the present disclosure, the composition described above, wherein the essence water is derived from one of a fruit, grass, bark, shrub and tree. According to the present disclosure, the composition described above, wherein the composition is one of a topical composition and an ingestible composition.

In an embodiment, plant matter from any of the following plants or plant parts is suitable for generating the essential oil and/or essence water: Angelica, Aniseseed, Basil, Bay, Benzion, Bergamot, Birch, Blue Tansy, Cabreuva, Cajeput, Calamus, Capsicum, Caraway, Cardamon, Carrot Seed, Cassia, Cedarwood, Celery Seed, Chamomile (German, Roman, Egyptian), Chaste Tree, Chaulmoogra, Cilantro, Cinnamon Bark, Cinnamon Berry, Cistus, Citronella Cylon, Citronella Java, Clary Sage, Clementine, Clove Bud, Copaiba Balsam, Coriander, Cumin, Cyprus (sempervirens, torulosa), Davana, Dill, Dong Qaui, Elemi, Eucalyptus (Blue Mallee, Globulus, Peppermint, Radiata), Fennel Sweet, Fenugreek, Fir (Idaho Balsam, Siberica), Frankincense (carterii, frereana, seratta), Galbanum, Garlic, Geranium, Ginger (CO2, Sweet), Gingergrass, Goldenrod, Grapefruit (pink, white), Helichrysum (italicum, angustifolia, absolute), Hinoki, Holy Basil, Howood, Hyssop, Idaho Tansey, Jasmine (grandiflorum, sambac), Juniper berry, Kanuka, Katrafay, Laurel, Lavender, Ledum, Lemon, Lemon Myrtle, Lemongrass, Lime, Litsea Cubeba, Mandarin, Manuka, Marjoram, Melissa (blend, pure, rectified), Mountain Savory, Myrrh, Myrtle, Neroli, Niaouli, Nutmeg, Opoponax sweet Myrrh, Orange (Bitter, Sweet, Sweet Dark), Oregano wild, Oregano, Osmanthus, Palmarosa, Pala Santo, Parsley, Patchouli, Peper (black, white), Peppermint (arvensis, piperita), Petitgrain, Pine Needle, Ravensara, Rose (Geranium, Absolute), Rosemary, Rosewood, Rue, Sage, Sandalwood, Saro, Spanish Sage, Spearmint, Spikenard, Spruce (picea, tusga), St. John's Wort, Tagette, Tangerine, Tarragon, Tea Tree, Tree, Thyme, Tumeric, Valerian, Vanilla, Vetiver, Violet Leaf, Wintergreen, Yarrow (Blue, Green) Ylang Ylang, Zanthoxylum, Malaleuca (quinquenervia,alternifolia), Laurus Nobilis, Jade Lemon, Kong Kuai, Dorado Azul.

According to the present disclosure, a method for the preparation of an essential oil and essence water mixture is provided, said method comprising: processing the essential oil to reduce a size of its components to small particles; and distributing the small particles of the processed essential oil throughout the essence water. According to the present disclosure, the method described above, further comprising deriving the essential oil from plant matter. According to the present disclosure, the method described above, further comprising wherein the step of deriving the essential oil from plant matter comprises directing stream through the plant matter. According to the present disclosure, the method described above, further comprising deriving the essence water from plant matter. According to the present disclosure, the method described above, further comprising wherein the step of processing the essential oil to reduce a size of its components to small particles comprises placing the essential oil under high pressure. According to the present disclosure, the method described above, further comprising wherein the step of processing the essential oil to reduce a size of its components to small particles comprises spinning the essential oil at high RPMs in a centrifuge. According to the present disclosure, the method described above, further comprising extracting the essential oil from a first type of plant matter and deriving the essence water from a second type of plant matter, wherein the first type of plant matter and the second type of plant matter are different. According to the present disclosure, the method described above, further comprising extracting the essential oil from a first type of plant matter and deriving the essence water from a second type of plant matter, wherein the first type of plant matter and the second type of plant matter are the same.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A composition for therapeutic treatment of a human being comprising:
an essential oil derived from plant matter;
essence water derived from plant matter;
wherein the essential oil and essence water form a homogeneous mixture.

2. The composition of claim 1, wherein the essential oil and the essence water are derived from different types of plants.

3. The composition of claim 1, wherein the essential oil and the essence water are derived from the same type of plant.

4. The composition of claim 1, wherein the plant matter from which the essential oil is derived is an herb.

5. The composition of claim 1, wherein the plant matter from which the essential oil is derived is a fruit.

6. The composition of claim 1, wherein the plant matter from which the essential oil is derived is bark from a plant.

7. The composition of claim 1, wherein the plant matter from which the essential oil is extracted is a grass.

8. The composition of claim 1, wherein the plant matter from which the essential oil is extracted is eucalyptus.

9. The composition of claim 1, wherein the plant matter from which the essential oil is extracted is one of a shrub and tree.

10. The composition of claim 1, wherein the essence water is derived from an herb.

11. The composition of claim 1, wherein the essence water is derived from one of a fruit, grass, bark, shrub and tree.

12. The composition of claim 1, wherein the composition is one of a topical composition and an ingestible composition.

13. A method for the preparation of an essential oil and essence water mixture, said method comprising:
processing the essential oil to reduce a size of its components to small particles; and
distributing the small particles of the processed essential oil throughout the essence water.

14. The method of claim 13, further comprising deriving the essential oil from plant matter.

15. The method of claim 14, wherein the step of deriving the essential oil from plant matter comprises directing stream through the plant matter.

16. The method of claim 12, further comprising deriving the essence water from plant matter.

17. The method of claim 13, wherein the step of processing the essential oil to reduce a size of its components to small particles comprises placing the essential oil under high pressure.

18. The method of claim 13, wherein the step of processing the essential oil to reduce a size of its components to small particles comprises spinning the essential oil at high RPMs in a centrifuge.

19. The method of claim 13, further comprising extracting the essential oil from a first type of plant matter and deriving the essence water from a second type of plant matter, wherein the first type of plant matter and the second type of plant matter are different.

20. The method of claim 13, further comprising extracting the essential oil from a first type of plant matter and deriving the essence water from a second type of plant matter, wherein the first type of plant matter and the second type of plant matter are the same.
